(19) United States
(12) Patent Application Publication
(10) Pub. No.: US 2005/0245846 A1
(43) Pub. Date: Nov. 3, 2005

(54) Vibrating, magnetically guidable catheter with magnetic powder commingled with resin, extruded as an integral part the catheter

(76) Inventor: Don Eward Casey, Columbus, OH (US)

Correspondence Address: DON E. CASEY, 406 Thurber drive west #9, Columbus, OH, 43215, US

(21) Appl. No.: 10/837,116

(22) Filed: May 3, 2004

(51) Int. Cl. A61B 5/00

(52) U.S. Cl. 600/585; 600/434; 604/523

(57) ABSTRACT

A catheter that is produced in which magnetic powder is commingled and becomes an integral part of the catheter, which makes it not only guidable within the body by an external device, but can be made to vibrate at various speeds (cycles per second) and at varying intensities (voltages) to not only prevent its adhesion to vascular walls, but to render plaque into a viscous state, for easy suctioning from the body.

### FIELD OF THE INVENTION

This invention relates to catheters, in particular to a manufactured catheter in which magnetic powder is mixed with the resin and extruded as an integral part of the catheter.

### BACKGROUND OF THE INVENTION

Prior art shows conventional magnetic catheters designed so that magnet(s), made in various shapes, are attached to the catheter after the catheter has been formed by a tube extrusion machine. These magnets typically take one of two shapes: A tubular distal (forward) tip affixed by various attachment methods to the distal end of a catheter for the purpose of guiding and-or pulling the catheter from its advancing end, addressing the problem of catheters lacking longitudinal stiffness when advanced from the proximal (trailing) end, without what is commonly known as a “J” wire inserted into and running through the catheter's lumen or hollow center.

The second design is for magnets to be compression molded as short, thin-walled sleeves or rings, slid over the outside of a catheter or inserted inside the lumen (U.S. Pat. No. 6,689,119 B1) and typically held in place by surgical adhesive. Outside positioned sleeve magnets can be placed anywhere along a catheter's length, providing a means of pulling or moving the catheter at intermediate points along its length by external magnets, since catheters develop adhesion to plaque that tends to coat the entire vascular walls to varying degrees of thickness.

### SUMMARY OF THE INVENTION

This invention comprises a vascular catheter with powdered rare-earth magnetic metal in very fine or granulated composition, possibly with other metal or non-metal substances, impregnated along all or part of the catheter's length in various densities at the time the catheter is formed from a tube extrusion machine, to achieve two results:

One: The placement of an induction coil emanating a pulsating (half-wave) d.c. or a full wave a.c. field, which can be widely varied in frequency and intensity (voltage), is placed alongside the patient's body, but not attached thereto, while the patient is lying on an operating table, creating what is called a sympathetic vibration of the catheter along its entire length while it is being inserted, preventing accumulative adhesion to the wall of the vasculature while it is being advanced to a blockage site. This coil is attached to a frequency converter electronic device which powers the coil. The fact that catheters vary in diameter from about 0.040″ inches down to 0.015″ inches means they have relatively little longitudinal stiffness, although presently the 0.015″ diameter catheters tend to be about a foot in length, mainly for insertion via the carotid artery in the neck and advanced to a location in the brain. Although it is true that the insertion of a “J” wire gives catheters stiffness, this stiffness acts adversely when the catheter needs to follow a circuitous pathway to reach a blockage site, typically found near the terminus of an artery, where the arterial wall is thin and subject to easy damage by perforation by the “J” wire.

Two: Placement of a two-sided pulsating (half-wave) d.c. or an a.c. powered coil can be selectively positioned so as to focus vibrations on the forward or distal tip when it arrives at the blockage site, making it vibrate at a frequency (cycles-per-second) and intensity (voltage) creating low friction heating, reducing plaque to a viscous state, rendering it quickly suctioned out of the body with the aid of a pump connected to the proximal end, outside the body. Prior art shows one or two patents in which a vibrating device is attached to the “J” wire, causing the entire wire to vibrate as a way to loosen plaque, which is not a good idea, because the suction pump can not withdraw plaque while the “J” wire blocks the lumen. Also, harmonics will be created along the approximately three foot long “J” wire which could create violent whipping action, if not breaking the wire.

This a.c. or half-wave pulsating d.c. powered coil will have a truncated cone affixed to one side, with sufficiently thick iron construction so that the cone does not vibrate and impart vibrations into the body, but serves only as a shunt, focusing the vibratory field of the coil so vibrations will pass through the narrow, truncated open end of the cone, vibrating only the distal tip of the catheter or that part where a concentration of vibrations is desired, while keeping a cardiac pacemaker that may be implanted close to the patient's heart, shielded. It is known in the literature regarding magnetic fields that there are two barriers to bar a magnetic field: One is simply air space or distance, the second is iron, which tends to act as a shunt or absorbing barrier to such a field. In fact, when rare earth magnets are shipped by common carrier, they are classified as “Hazardous Cargo,” and must be positioned in the center of an outer steel (iron) cage or box within an outer shipping container, to provide magnetic isolation. The coil is designed so that either the open or the cone-shaped side can face toward the patient's body, or held by a technician, so the entire catheter will vibrate while it is being inserted and guided to the blockage site, then by flipping over the coil, only a portion of the forward portion of a catheter will vibrate. When the catheter has reached the blockage site, the coil can be repositioned so its cone side is facing toward the distal tip of the catheter, which is being continuously viewed with the aid of a fluoroscopic or an ultra-sound machine, each machine having advantages with regard to looking inside the body: Fluoroscopic machines (weak x-rays) can see through bones and vacant space (lungs); whereas, ultra sound machines can be positioned to “see” between bones such as ribs, and can view in false coloring, and adjusted to view only to a pre-set depth within the body, minimizing screen clutter.

This new catheter can also be guided to the blockage site by both a “J” wire and an external magnetic guider, said “J” wire may have any number of pre-bends at its distal tip so as to aid in its steerage through a tortuous passageway of the vasculature.

However, this design calls for another guidance method not requiring the “J” wire. The magnetic powder impregnated into the catheter wall allows the catheter to be guided by an external, hand-held magnetic guiding device. This hand-held guider (to be discussed in a separate application) with its magnets positioned close to the body, obviates the need for giant magnetic fields created by very large electro-magnetic coils, as shown in Stereotaxis and other patents regarding this subject area. All magnetic fields drop off dramatically (at a non-linear rate) as their distance is increased from another magnet or magnetically responsive metal. Therefore, a magnetic field positioned very close to the body will not have to be nearly as powerful. It should also be noted that non-magnetic metals, such as Nitinol, may also be used in this catheter, however, such a catheter will not be nearly as responsive to an external magnetic field.

Another feature of this catheter permits other metals to be commingled and extruded into a catheter at the moment of its manufacture. Such a metal might be Nitinol, having memory restoration and retention qualities that can be useful, especially if Nitinol metal particles are of a sufficient elongate length and positioned parallel to the catheter's length. This positioning will naturally occur, because of the thin walls of the catheter.

Another feature of this invention includes medicine in solid, particulate form, being commingled and extruded with the catheter so that the drug is protruding onto the surface of the catheter, subsequently eluted into the walls of the vasculature at the time of catheter insertion and destination positioning, which will be released from the catheter walls due to vibrations of the catheter. Such drugs will protrude onto the surface of the catheter in the same manner as metal particulate shown in FIG. 3 for abradant purposes is protruding on the catheter surface.

Another feature of this invention is the coating of the catheter's outside walls or inside walls, such as a clotting chemical, which will tend to clot vasculature walls should the catheter cause trauma to said walls.

### BRIEF DESCRIPTION OF THE DRAWINGS

FIG. 1 A view of a catheter showing finely granulated rare-earth magnetic powder or other metals or medicine impregnated into as an integral part of the catheter, which can have a funnel shaped tip, or a rounded one.

FIG. 2 A view of the distal end of a catheter showing a greater density of magnetic powder near the distal tip, with a lesser concentration along its remaining length.

FIG. 3 A view showing magnetic powder or solid particulate drugs coarsely ground and sharp edged, so that it is larger in particle size, causing the particles to protrude on the surface of the catheter wall, providing an abradant surface when the catheter is vibrated by an external means, which can also transmit a drug into vascular walls.

FIG. 4 A view showing pulsating, (half-wave) d.c. or a full wave a.c. coil featuring a truncated cone affixed to one side, narrowing the d.c. or a.c. induced vibratory field by positioning the broad or by positioning the open side facing the patient.

FIG. 5 A view showing the frequency converter coil's truncated side facing a patient, attached to a frequency generator-converter device.

FIG. 6 A view showing an insertable reduced diameter distal tip so as to be positioned inside a magnetically responsive vascular stent, both members being magnetically attracted to keep the stent attached to the catheter until reaching the placement site in the vasculature, where it is repelled from the catheter by an external magnetic field.

Reference numerals in the drawings correspond to reference numerals in the text.

### DETAILED DESCRIPTION OF THE INVENTION

The first embodiment of this invention shown in FIG. 1, numeral 1 highlights the elegant simplicity of this design over prior art showing rare-earth or (Alinco-ceramic, etc.) magnetic powder or more common metals impregnated into the resin, in the tube extrusion machine in various densities and shapes, before or as the catheter is extruded, making the powder an integral part of the catheter, not subject to separation from the catheter as is the case with magnets variously attached, after it has been extruded. This integral incorporation of magnetic powder is highly desirable over the use of separate magnets shown in other patents attached to the catheters, usually by surgical adhesive or wire in the case of magnets attached to the distal tip, because magnetic powder tends not to maintain good shape integrity. It tends to crumble and return to a powdered state, especially when subject to vibrations, as this catheter is designed to be. Rare Earth magnets, usually made of alloys consisting of Neodymium, Cobalt, Boron, Iron, and other additives, are up to fifty times stronger than typical ceramic magnets, commonly known as “refrigerator magnets.” Even when magnets are plated with a plastic resin, Nickel, Chromium, Gold or other materials, such thin platings tends to split and separate when the magnet is subjected to rough contact or vibrations. And when a catheter is inserted into the vasculature, flexing and bending of the catheter is inevitable, making expansion or compression stress on attached magnet(s) unavoidable. An obvious place where bending of a catheter first occurs upon insertion is at the top of the Aorta, where it must curve 180 degrees before entering the heart.

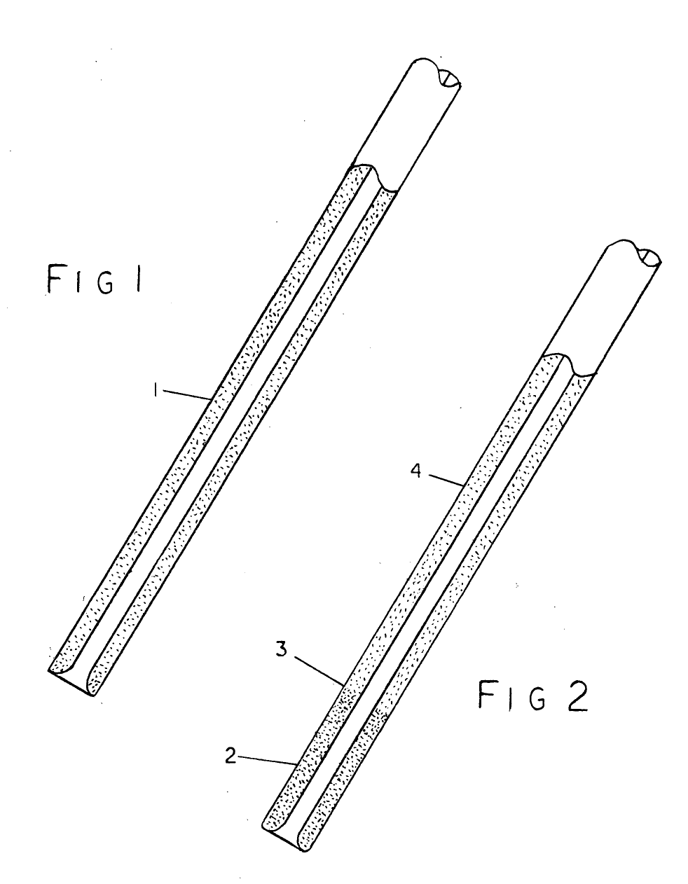

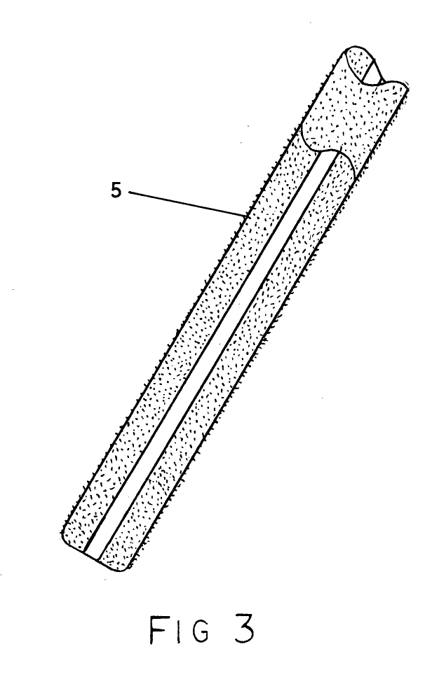

FIG. 2 Shows the result of a tube extrusion machine varying the density of metal powder as the catheter is being extruded, so as to concentrate power at the distal tip 2 for more authorative guidance. Reference numeral 3 shows a demarcation plane between powder heavily concentrated at the tip of a catheter, with reference 4 showing the magnetic powder at a lesser concentration level in the remainder of a catheter.

FIG. 3 Reference 5 illustrates the fact that magnetic powder can be granulated so as to have a round ball-like surface of any diameter desired, or be ground so it has a sharp surface texture that can protrude along the outside surface of the catheter, acting as an abradant when the catheter is made to vibrate, adding in the dislodgement of plaque on vascular walls. This protrusion of particulate can also consist of medicine in soluble particulate form that is imparted into the vascular walls as a result of these vibrations.

The primary purpose of having magnetic powder impregnated along the entire length of a catheter is so the entire catheter can be made to vibrate sympathetically with electronic emanations of an external half-wave d.c. or an a.c. pulsing field, via a coil attached to the output side of a variable frequency converter machine or a constant-frequency supply source. Simply stated, these machines (one made by Pacific Power) convert the 60 cps a.c. current emanating from wall outlets into a frequency of up to 5,000 cps or down to 25 cps by digitally dialing in the desired vibratory frequency and the desired intensity of these vibrations (voltage). Other converters have a vibratory output exceeding 8,000 cps, which means they can produce heat in the catheter at the temperature desired.

Making the catheter vibrate accomplishes two crucial tasks: While the catheter is being inserted and guided to the blockage site, it rubs against the adhesive walls of the vasculature, and due to plaque coating the entire vasculature in varying amounts, which produces accumulative adhesion. Given the fact that catheters have poor longitudinal stiffness, even with “J” wires inserted through their lumen (hole), preventing their clinging to arterial walls is important. This type of catheter can obviate the need to manipulate a patient by turning them on their sides, pounding on their rib cages or having them cough repeatedly to cause a catheter to curve in a desired direction.

Second, once the catheter is at the blockage site, making the distal tip vibrate at high cycles per second, and at the appropriate intensity (voltage) reduces plaque or blood clots into a viscous or semi-liquid state, permitting fast suctioning from the body, which is necessary to prevent the catheter from causing a heart attack or stroke, the greatest risk of using catheters. The second risk of using catheters in thin-walled blood vessels is that the “J” wire or guiding wire can accidentally punch through the wall, causing internal bleeding. It is useful to understood that a “J” wire does not attached to the catheter, but is inserted through the lumen for guidance purposes only, then is pulled out completely when its guidance function has been achieved so the suction pump can be turned on.

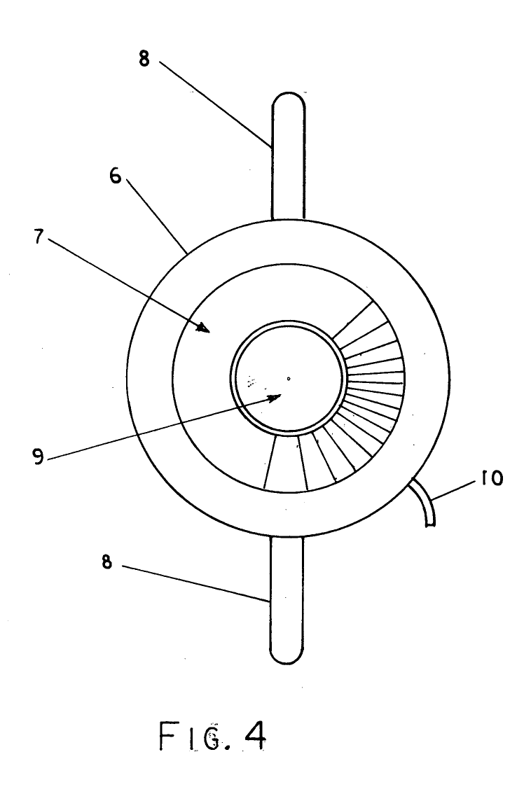

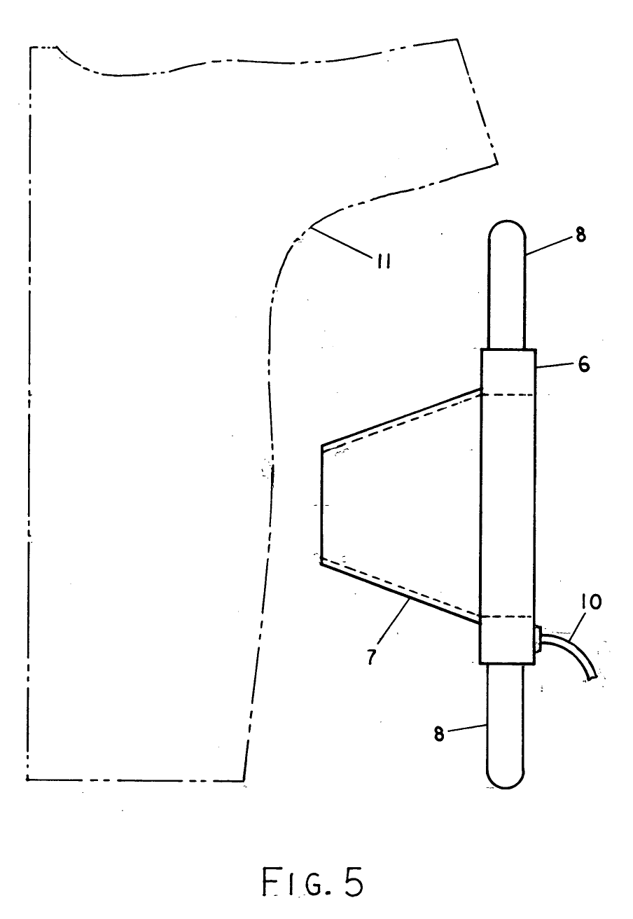

FIG. 4 shows the cone side of the coil to which d.c. or a.c. current can be fed from a power supply. Reference 6 is the outside top edge of an iron truncated funnel-type structure, inside of which houses the coil. Reference 7 shows longitudinal strakes or vibrational dampeners on the wall of this tapering housing, which can be placed inside or outside the cone, with reference 8 representing two handles, their knurled surfaces not shown, on opposite sides of the device, which the surgeon can use to grip and position the device. Note longitudinal strakes or raised ridges on the outside of this tapering coil housing serving as dampeners against the coin becoming a part of the vibratory field. Reference 9 shows the hole through which primary magnetic emanations will pass, unobstructed. Thus, when this truncated cone is facing the patient's body, magnetic emanations will be substantially (but not completely) absorbed by the iron cone, limiting the magnetic field to that passing through the hole in the cone. It should be noted that making this cone out of steel with a high iron content is necessary to serve as a magnetic shunt. Reference 10 shows the power cord running from this coil device to the power supply. The primary need for such a truncated cone is when the plaque removal procedure is used on patients wearing a cardiac pacemaker, defibrillator, and other implanted electronic devices. This will minimize the field in the immediate vicinity of the pacemaker. A pacemaker's Titanium case acts as a fairly good barrier for magnetic emanations, but not a complete one. In fact, what is called a magnetic “donut” is laid directly atop an implanted pacemaker, sending discrete magnetic pulses into the pacemaker to change pulse rate, width (duration) of the pulse, and the voltage traversing the lead wire to the patient's heart. However with the frequency converter to be used with this device, the cycles per second will be far above those used to re-pace a pacemaker.

FIG. 5 shows an overhead side view of the vibratory coil device positioned alongside a patient 11, below the left arm, a logical position while a catheterization procedure is being performed on the heart. Since the coil will not block the fluoroscopic view. Said coil device can also be hand-held by an assistant so as to better focus the vibratory field at the distal tip now within the body, however, the vibratory reach of the coil, based on tests, exceeds two feet, which means the patient's arm may be alongside their body, if that is desired. The other reference numbers were discussed in FIG. 4

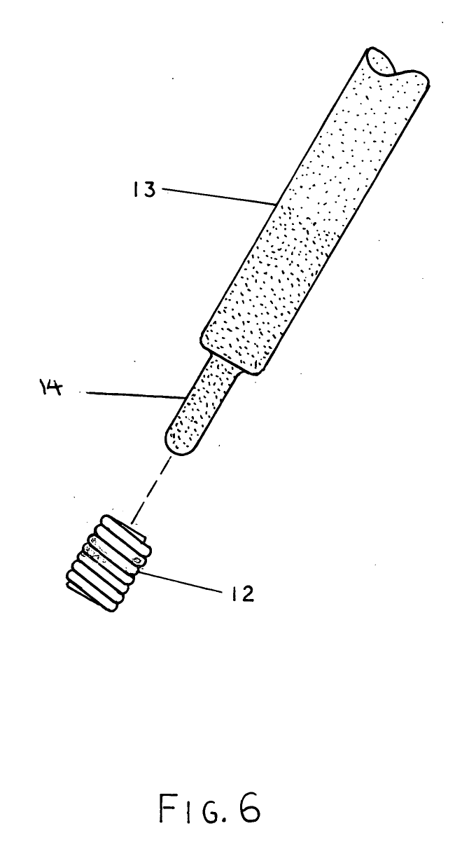

FIG. 6 Shows the forward or distal tip of a solid core (no lumen hole) catheter 13 with a reduced diameter tip 14 onto which a typical stent 12 can be slipped over and held in place by the attracting force of the magnetic powder within the catheter tip. The magnetic catheter for a stent placement procedure will not require a hole running through its center. In this figure, the stent is shown after it has been magnetically repelled from said tip by the external magnetic guider (to be revealed in a separate application). It is common knowledge within the field that stents are made in various shapes, some a simple coil-design as shown here, some having what is called a double-helix design, or simply a perforated tube, all having a common aspect involving openings along their length, and being flexible, to conform to the shape of a vascular passageway where it is placed.

Magnetic powder impregnated into the walls of a catheter can be magnetized so as to be positively or negatively charged (oriented) before or after the resin of the catheter hardens. This means the tip of this catheter can be charged upon its manufacture so it will attract and hold to its distal end a stent slipped over the tip before insertion into the body, if the stent is made of a magnetically responsive metal or coated with a magnetic paint, without the stent necessarily being magnetically charged (oriented). The stent can, upon reaching the intended placement site, be repelled from the catheter's distal tip at the same time the catheter is being pulled back by the technician holding the opposite, proximal end outside the body, aided by an external magnetic guider designed to operate with this invention, the same guider that pulls the catheter through the vasculature to the blockage.

As has been mentioned, this catheter can also be used in conjunction with a conventional “J”, even though said guidance wires are made with several different shaped tips. The advantage of not using a “J” wire with this or any other catheter is that it must be removed before the external suction pump can be switched on, since they block the lumen. And since the duration of catheter insertion at the blockage site of a small artery must be brief, to avoid inducing a heart attack or stroke, it will be desirable not to use a guidance wire, which this catheter is designed to not require. However, the option exists.

Regarding the placement of small magnets inside the lumen of a catheter, it must be understood that their placement will partly block the lumen, making it more difficult for the use of a “J” wire. As such a wire is inserted through the narrow lumen, the tip of this wire will tend to become caught on or blocked by any internally placed magnets. Also, the question of how to attach (glue) the magnets deep inside the lumen will produce doubtful results, at best. Inserting a tubular magnet deep inside the lumen by an inserter rod will rub off most if not all of the adhesive before it reaches the intended placement location, a problem not given sufficient thought by holders of such patents. Also, any adhesively attached magnet on the outside or the inside of a catheter, which is not an integral part of the resin, raises the possibility that such magnets will become fragmented and unattached, becoming free floating objects in the vascular system. Such free-floating, unattached magnets would require surgical removal, a problem this design completely avoids. Another consideration concerning insertion of magnetic sleeves on the outside or inside of catheters is that the volume of a magnet will not be great enough, without creating an unwanted degree of stiffness if it were made long or by placing many on the catheter, as a long sleeve or multiple sleeves will do.

However, injecting magnetic powder so it is an integral part of the entire catheter means the volume of magnetic material can be twenty times greater than catheters with attachable magnets, without creating unacceptable stiffness or breaking off. With this catheter, the amount of magnetic powder (by volume), can vary between 10% to as high as 90%, provided the catheter is not required to make sharp bends, before reaching the limit of tolerable stiffness. Also, the fineness of such magnetic powder can approach that of talcum powder, if desired. Anyone familiar with cassette tapes or VCR tapes knows how smooth the surface of such tapes is, even though the tape contains iron oxide powder. So, either a smooth or a rough catheter surface can be produced, depending which is desired. And, surface smoothness or roughness can be varied within a catheter.

Regarding guiding this catheter, particularly along the front side of the heart, it is useful to remember that the front of the heart is rarely more than three inches beneath the outside surface of a patient's chest, even in the case of obese or otherwise excessively developed individuals, meaning this catheter will be within easy magnetic “reach” of powerful external guider magnets moving over the surface of the chest or backside. Our tests have shown that the effective magnetic “reach” of two-inch square rare-earth magnets is about eight inches, which means not only the femoral artery but the aorta, running up the back of the torso, will be within easy magnetic “reach” of a surface positioned external guider. Regarding catheter guidance to locations within the brain, the maximum distance from outside-positioned surface magnets will not exceed four inches, based on this researcher's above sized cranium.

Regarding coating this catheter with an additional chemical, such as one that induces clotting of the blood, the result of bleeding a catheter may induce, such coating will be highly desirable. Such bleeding of vascular walls is more likely in older patients where the walls are flaccid and weakened, from age, which is a typical condition of people most likely in need of plaque removal. Also, the catheter's outside surface coating may consist of a chemical that has a dilating effect on the vasculature, holding the walls open farther during this procedure.

Regarding a comparison between this plaque removal procedure and the long used angioplasty or balloon procedure, the following applies: The balloon method is primarily designed to compress or radially expand (push aside) vascular wall plaque when air pressure or a liquid inflates the distal balloon tip, temporarily opening the passageway, while removing little if any plaque. The result of this treatment is that the artery tends to reclose within six months to five years, requiring the angioplasty procedure be repeated, exposing the patient to the risk of a catheter-induced heart attack or stroke.

Regarding the catheter shown in U.S. Pat. No. 6,524,303 B1 describing the insertion of a second catheter inside an outer catheter, the following applies: The amount of surface to surface contact between the two catheters introduces the problem of accumulative suction or adhesion between the two tubes. The inner catheter would resist differential movement with the outer catheter tube.

And, placing an extremely small coil at the distal tip as shown in patents (U.S. Pat. Nos. 6,304,769 B1 & 6,375,606 B1), given the fact that a catheter's outside diameter tends to average only 0.025″ inches, with wall thickness not exceeding 0.010″ inches, means the wire used to wind such a coil will have to be extremely small, subjecting it to easy breakage. Secondly, the limited number of turns in such a small coil will preclude inducement of a strong electromagnetic field, since an emf field is directly proportional to the number of turns in a coil, plus the wire's ability to handle amperage load. Thirdly, when viscous resin (the thickness of toothpaste) is injected through an extrusion die with a force between 400 to 800 pounds per square inch, the turns of a fine wire coil would be distorted or be jammed together, not remain neatly spaced turns, as shown in those patent drawings. Also, if both ends of a coil wire are run back inside the catheter to an outside of body power supply, this coil wire will have to be around 0.001″ inch in diameter, to be embedded in the thin walls of a catheter. This researcher has worked with extremely small diameter coil wire on a project building transmitters placed on the backs of small insects, and is familiar with the limitations of small diameter magnet wire, sometimes referred to as “spider web wire.”

From an overview of prior art, it is clear that those patents showing the placement of small coils embedded within a catheter, the coil will be ineffective in generating a significant electromagnetic field. And, slidable catheters placed inside an outer catheter are impractical because the inner one will adhere to the outer one, and will further reduce the lumen diameter of the inner catheter, rendering it ineffective and subject to collapsing. This collapsing of the catheter(s) will occur most likely where the catheter makes sharp bends.

1. A catheter for use in a body, comprising
a catheter having
a distal end,
and a middle section contiguously disposed between and joining said distal end, said proximal end;
integrated magnetic material, said magnetic material permeating said distal end, said proximal end, and middle section of the catheter;
an external guiding means, comprising a source of a controlled and directed magnetic field, said controlled and directed magnetic field being directed towards said catheter for manipulating the position and motion of said catheter;
said integrated magnetic material being responsive to and cooperating with said external magnetic guiding means;
means for observing and reporting the position of the catheter within the body;
and, operator means employing said observing and reporting means, for directing and controlling the position and motion of the catheter in the body.

2. The catheter system as described in claim 1, wherein said permeating magnetic material is disposed in uniform density across said distal end, said middle section, and said proximal end.

3. The catheter system as described in claim 1, wherein said permeating magnetic material is disposed in non-uniform density across said distal end, said middle section, and said proximal end.

4. A catheter system as described in claim 1 wherein said distal end further comprises a means for holding a vascular stent at said distal end, and for preventing movement of the vascular stent from said distal end and onto said middle section; and means for expelling the vascular stent from said distal end upon command of said operator.

5. A catheter system as described in claim 1 wherein said distal end, said middle section, or said proximal end further comprise an abrading means for dislodging matter attached to a vascular wall.

6. A catheter system as described in claim 1 wherein said distal end comprises a means for scooping matter that is encountered in the vasculature.

7. A catheter system as described in claim 1 wherein said distal end further comprises a generally smoothly rounded forward surface for navigation through a vasculature.

8. A catheter system as described in claim 1 wherein said distal end, said middle section, or said proximal end further comprises means for delivering a medicine to a body area wherein said medicine comprises a solid particulate matter disposed on said distal end, said middle section, or said proximal end.

9. The catheter system as described in claim 8, wherein said permeating magnetic material is disposed in uniform density across said distal end, said middle section, and said proximal end.

10. The catheter system as described in claim 8, wherein said permeating magnetic material is disposed in non-uniform density across said distal end, said middle section, and said proximal end.

11. A catheter system as described in claim 8 wherein said distal end further comprises a means for holding a vascular stent at said distal end, and for preventing movement of the vascular stent from said distal end and onto said middle section; and means for expelling the vascular stent from said distal end upon command of said operator.

12. A catheter system as described in claim 8 wherein said distal end, said middle section, or said proximal end further comprise an abrading means for dislodging matter attached to a vascular wall.

13. A catheter system as described in claim 12 wherein said distal end comprises a means for scooping up matter that is encountered in the vasculature.

14. A catheter system as described in claim 8 wherein said distal end further comprises a generally smoothly rounded forward surface for navigation through a vasculature.

15. A catheter system wherein the outside surface of the catheter is coated with a chemical which has a dilating effect on the vasculature.

16. A catheter system as described in claim 15 wherein the chemical coating on the outside surface of a catheter has a clotting effect on fluid seepage from the vascular walls.

17. A catheter system in which electromagnetic vibrations from a non-attached source is of sufficient intensity so as to vibrate and heat all or part of a catheter.

18. A catheter system in which the magnetic particulate is embedded in the resin of the catheter so as to be oriented with regard to its direction of orientation.
